(19) United States
(12) Patent Application Publication
(10) Pub. No.: US 2022/0022848 A1
(43) Pub. Date: Jan. 27, 2022

(54) ULTRASOUND IMAGING SYSTEM USING COHERENCE ESTIMATION OF A BEAMFORMED SIGNAL

(71) Applicant: UNIVERSITY OF SOUTHERN CALIFORNIA, Los Angeles, CA (US)

(72) Inventors: Jesse Tong-Pin Yen, La Crescenta, CA (US); Yang Lou, San Antonio, TX (US)

(21) Appl. No.: 17/382,173

(22) Filed: Jul. 21, 2021

Related U.S. Application Data
(60) Provisional application No. 63/055,743, filed on Jul. 23, 2020.

(51) Int. Cl. A61B 8/08; G01S 15/89; G01S 7/288; A61B 8/00

(52) U.S. Cl. CPC A61B 8/5207; G01S 15/8925; A61B 8/4483; A61B 8/4444; A61B 8/461; G01S 7/2883

(57) ABSTRACT

Improved ultrasound imaging using coherence estimation of a beamformed signal. Ultrasound imaging using coherence estimation of a beamformed signal as described herein may be performed by applying a plurality of filters to the beamformed signal to generate a plurality of filtered beamformed signals. Normalized cross-correlation may be performed on a plurality of pairs of filtered beamformed signals to determine a coherence coefficient corresponding to each pixel of an ultrasound image, which may be used to construct a coherence estimation ultrasound image.

### CROSS-REFERENCE TO RELATED APPLICATIONS

This application claims priority to and the benefit of U.S. Provisional Patent Application No. 63/055,743, titled “ULTRASOUND IMAGING SYSTEM USING COHERENCE ESTIMATION OF A BEAMFORMED SIGNAL,” filed on Jul. 23, 2020, and the entirety of which is hereby incorporated by reference herein.

### BACKGROUND

### 1. Field

The present disclosure relates to ultrasound imaging systems, particularly improved ultrasound imaging systems using coherence estimation of a beamformed signal, and methods of using the same.

### 2. Description of the Related Art

Ultrasound images are traditionally constructed primarily from the amplitude of the received echo. Strong reflections result in a high amplitude echo which is mapped to white on the screen. No reflected echoes are mapped to black. However, this traditional method results in incoherent echoes from acoustic clutter, noise, and sidelobes, reducing the contrast of the resulting image. For example, in echocardiography, the visualization of heart chambers and nearby blood vessels may be difficult due to poor ultrasound image contrast. These problems may be exacerbated if the patient is obese, but these patients may benefit the most from a high quality, high contrast echocardiogram. It has been estimated that 10-15% of images are suboptimal or non-diagnostic. In another example, in breast ultrasound, a common task is to differentiate solid and cystic masses. Simple anechoic cysts with fill-in caused by multiple scattering, reverberations and clutter can be misclassified as benign masses or malignant lesions. Levels of fill-in are increased in the presence of aberrations caused by intermittent layers of fat and tissue. Delineation of carcinoma may also be improved with better signal processing methods that improve contrast. In yet another example, in abdominal ultrasound, imaging of gallbladder stones and polyps, assessment of thrombus or plaque in the aorta, solid organ and transplant vasculature, and differentiation between simple cysts, complicated cysts and solid nodules within organs such as the liver, spleen and pancreas may be improved with improved contrast. For hepatic imaging, visualization of cystic liver lesions and dilated bile ducts may be improved.

Some coherence-based methods of constructing ultrasound images, such as generalized coherence factor (GCF), phase coherence factor (PCF), sign coherence factor (SCF), and short-lag spatial coherence (SLSC) have previously been investigated. A coherence factor (CF) uses the ratio of the coherent energy to the total incoherent energy of the received radio frequency (RF) data to weigh each image point at every depth. GCF was developed by modifying CF to account for the energy spread by speckle-generating targets that CF does not take into consideration. The received RF signals from the main lobe region are coherent and correspond to low frequency components, whereas those from sidelobe and clutter are incoherent and correspond to high-frequency components, GCF is computed as a ratio of the spectral energy within a low frequency region to the total spectral energy. A matrix of GCF values is then used to weigh each pixel within the field-of-view (FOV). PCF and sign coherence factor SCF employ a sidelobe reduction approach similar to GCF, but the pixel-by-pixel weighing is based on phase diversity of the delayed channel RF signals across the aperture rather than coherence. SLSC forms images similar to conventional B-mode images using lateral spatial coherence as the basis of image formation. SLSC methods use channel data combined with a measure of similarity among the channel data to estimate coherence. Once the estimate of coherence is obtained, the estimate may be multiplied to the conventional B-mode image or used as an image itself. A commonality of these methods is that they use channel data to estimate coherence. Collecting, transferring, storing, processing, and cross-correlating channel data may be cumbersome and time consuming.

Hence, there is a need for an ultrasound system that produces a high quality and high contrast image and relies on a single beamformed data to estimate coherence instead of multiple channel data.

### SUMMARY

Examples described herein relate to embodiments of improved ultrasound imaging systems using coherence estimation of a beamformed signal, and methods of using the same. In some embodiments, the systems and methods described herein may be used to construct improved ultrasound images using coherence of a beamformed signal. In some embodiments, the improved ultrasound images may have higher contrast-to-noise and/or signal-to-noise ratios compared to traditional ultrasound images, and these improvements may be achieved without need to perform coherence estimation techniques on individual transducer channel (e.g., element) data. Additionally, although many embodiments of the systems and methods described herein estimate coherence of beamformed signals, in certain embodiments coherence estimations may be performed on ultrasound signal data that is not beamformed. For example, in some embodiments, a single element is used to receive signals, so it may not be necessary to beamform the reflected signal before using coherence estimation to construct an improved image. In other embodiments, it may be desirable to use coherence estimation of channel data that has not been beamformed. Embodiments of the systems and methods described herein have several features, no single one of which is solely responsible for their desirable attributes.

In one aspect, the invention is embodied in a method of ultrasound imaging using coherence estimation which includes receiving beamformed ultrasound signals by a processor. The method includes assembling the beamformed ultrasound signals into an RF signal matrix by the processor. The method includes generating multiple filtered RF signal matrices using multiple spatial filters and the RF signal matrix by the processor. The method includes performing a normalized cross-correlation on multiple pairs of the filtered RF signal matrices to determine multiple cross-correlation coefficients corresponding to each pixel in an image of a target by the processor. The method includes determining a coherence coefficient using the multiple cross-correlation coefficients by the processor. The method includes constructing a coherence estimation image of the target using the multiple coherence coefficients corresponding to each pixel by the processor. The method includes displaying the coherence estimation image of the target on a display by the processor.

These and other embodiments may optionally include one or more of the following features. The method may include transmitting an ultrasound signal towards an imaging target using an array of ultrasound transducer elements. The method may include receiving multiple reflected ultrasound signals using the array of ultrasound transducer elements. The method may include beamforming the multiple ultrasound signals using the array of the ultrasound transducer elements.

The method may include transforming the RF signal matrix into a k-space representation of the RF signal matrix using a frequency transform to generate the multiple filtered RF signal matrices. The frequency transform may be a 2D Fast Fourier Transform (FFT) or other n-dimensional FFT.

Generating the multiple filtered RF signal matrices may include multiplying the k-space representation of the RF signal matrix by the multiple spatial filters to generate multiple k-space representations of the multiple filtered RF signal matrixes. The method may include transforming the multiple k-space representations of the multiple filtered RF signal matrices into time domain representations of the multiple filtered RF signal matrices using an inverse frequency transform.

In another aspect, the invention may be embodied in a computer readable medium storing program instructions. The program instructions include operations that include receiving multiple beamformed ultrasound signals and assemble the multiple beamformed ultrasound signals into an RF signal matrix. The program instructions include generating multiple filtered RF signal matrices by applying multiple spatial filters to the RF signal matrix. The program instructions include performing normalized cross-correlation on multiple pairs of the filtered RF signal matrices to determine multiple cross-correlation coefficients corresponding to each pixel in the image of the target. The program instructions include determining a coherence coefficient using the cross-correlation coefficients. The program instructions include constructing a coherence estimation image of the target using the coherence coefficients corresponding to each pixel. The program instructions include displaying the coherence estimation image of the target on a display.

These and other embodiments may optionally include one or more of the following features. The program instructions may include transmitting an ultrasound signal towards an imaging target using an array of ultrasound transducer elements and receiving multiple reflected ultrasound signals using the array of ultrasound transducer elements. The program instructions may include beamforming the multiple ultrasound signals.

The program instructions may include transforming the RF signal matrix into a k-space representation of the RF signal matrix using a frequency transform. The program instructions to generate the multiple filtered RF signal matrices may include generating multiple k-space representations of the multiple filtered RF signal matrices by multiplying the k-space representation of the RF signal matrix by the multiple spatial filters. The program instructions may include transforming the multiple k-space representations of the multiple filtered RF signal matrices into time domain representations of the multiple filtered RF signal matrices using an inverse frequency transform.

In another aspect, the invention is embodied in an ultrasound imaging system using coherence estimation. The ultrasound imaging system includes a display screen. The ultrasound imaging system includes an ultrasound probe having an array of transducer elements. The ultrasound probe is configured to transmit a beamformed ultrasound signal towards an imaging target using the array of transducer elements. The ultrasound probe is further configured to receive multiple reflected ultrasound signals using the array of transducer elements. The ultrasound probe is further configured to beamform the multiple received ultrasound signals using the array of transducer elements. The ultrasound imaging system includes a memory configured to store data. The ultrasound imaging system includes a processor coupled to the memory. The processor is configured to assemble the beamformed ultrasound signal into an RF signal matrix. The processor is further configured to generate multiple filtered RF signal matrices using multiple spatial filters and the RF signal matrix. The processor is further configured to perform normalized cross-correlation on multiple pairs of the filtered RF signal matrices to determine multiple cross-correlation coefficients corresponding to each pixel in an image of the imaging target. The processor is further configured to determine a coherence coefficient corresponding to each pixel in the ultrasound image of the target using the multiple cross-correlation coefficients. The processor is further configured to construct a coherence estimation image of the target using the coherence coefficients corresponding to each pixel. The processor is further configured to display the coherence estimation image on the display screen.

These and other embodiments may optionally include one or more of the following features. The coherence estimation image may have a higher contrast-to-noise ratio than an ultrasound image constructed from an amplitude of a received echo. The coherence estimation image may have a higher signal-to-noise ratio than an ultrasound image constructed from an amplitude of a received echo.

Performance of the normalized cross-correlation may be on segments of data approximately 1-4 wavelengths long in an axial direction for each filtered RF matrix in a given pair of filtered RF signals. Forming the coherence estimation image may include using a grayscale with a coherence coefficient of 0 mapped to total black and a coherence coefficient of 1 mapped to total white.

In some embodiments, determining the coherence coefficient for a given pixel may include summing the cross-correlation coefficients for the given pixel. In some embodiments, determining the coherence coefficient for a given pixel may include weighing and summing the cross-correlation coefficients for the given pixel. In some embodiments, determining the coherence coefficient for a given pixel may include averaging the cross-correlation coefficients for the given pixel.

### BRIEF DESCRIPTION OF DRAWINGS

Other systems, methods, features, and advantages of the present invention will be apparent to one skilled in the art upon examination of the following figures and detailed description. Component parts shown in the drawings are not necessarily to scale and may be exaggerated to better illustrate the important features of the present invention.

FIG. 1 is a schematic diagram of an example ultrasound imaging system using coherence estimation of a beamformed signal and an ultrasound processing system (UPS) for coherence estimation of a beamformed signal according to an aspect of the present disclosure;

FIG. 2 is a process flow diagram for an example process of improving ultrasound imaging using the ultrasound imaging system of FIG. 1 that applies coherence estimation to a beamformed signal according to an aspect of the present disclosure;

FIG. 3 is a process flow diagram for an example process of improving ultrasound imaging using coherence estimation of a beamformed signal according to an aspect of the present disclosure;

FIG. 4A illustrates a transmit-receive convolutional process with an aperture width of D yielding a lateral k-space function having a triangle function with a width 2D according to an aspect of the present disclosure;

FIG. 4B illustrates a transmit-receive convolutional process with a transmit aperture of width D and a receive aperture of an element approximated as a delta function yielding a lateral k-space function that is a rectangle function of width D centered at x₁ according to an aspect of the present disclosure;

FIG. 5 illustrates obtaining an estimate of an element response in k-space by dividing a single element response by an overall k-space response of a full aperture according to an aspect of the present disclosure;

FIG. 6 shows an embodiment of a k-space representation of a spatial filter for use in ultrasound imaging using coherence estimation of a beamformed signal according to an aspect of the present disclosure;

FIG. 7 shows example k-space representations of a set of three spatial filters overlapping in the axial and lateral directions, for use in ultrasound imaging using coherence estimation of a beamformed signal according to an aspect of the present disclosure;

FIG. 8 is an example plot of the contrast-to-noise ratios (CNR) achieved by the SLSC technique and embodiments of the coherence estimation using beamformed signals (Lateral and Axial Coherence Estimation, LACE) described herein, as a function of the percentage of overlap of filters used for each technique, modeled using the Field II simulation program according to an aspect of the present disclosure;

FIG. 9 is a plot of the signal-to-noise (SNR) ratios achieved by the SLSC technique and the LACE technique as a function of the percentage of overlap of filters used for each technique, modeled using the Field II simulation program according to an aspect of the present disclosure;

FIG. 10 is a plot of the CNR for embodiments of the coherence estimation methods described herein as a function of number of filters applied (N), modeled using the Field II simulation program according to an aspect of the present disclosure;

FIG. 11 is a plot of the SNR for embodiments of the coherence estimation methods described herein as a function of N, modeled using the Field II simulation program according to an aspect of the present disclosure;

FIG. 12A is a set of ultrasound images generated using the Field II simulation program of delay-and-sum (DAS), SLSC, and LACE according to an aspect of the present disclosure;

FIG. 12B is a set of ultrasound images generated using the Field II simulation program of DAS, SLSC, and LACE according to an aspect of the present disclosure;

FIG. 12C is a set of ultrasound images generated using the Field II simulation program of DAS, SLSC, and LACE according to an aspect of the present disclosure;

FIG. 13A shows a series of in vivo images of a gall bladder processed using traditional DAS, traditional SLSC, and LACE with 80% overlap according to an aspect of the present disclosure;

FIG. 13B shows a series of in vivo images of a heart in the 4-chamber apical view processed using traditional DAS, traditional SLSC, and LACE with 80% overlap according to an aspect of the present disclosure;

FIG. 14 shows an embodiment of a k-space representation of an estimated spatial filter for use in ultrasound imaging using coherence estimation of a beamformed signal according to an aspect of the present disclosure;

FIG. 15A shows an embodiment of a k-space representation of a beamformed channel point spread function (PSF) according to an aspect of the present disclosure;

FIG. 15B shows an embodiment of a k-space representation of a channel PSF according to an aspect of the present disclosure;

FIG. 15C shows an embodiment of a k-space representation of estimated spatial filters where spatial filters may be estimated from the ratio between the k-space representations of the channel PSF and the beamformed PSF, and the spatial filters may be divided into several axial segments for optimal estimation according to an aspect of the present disclosure;

FIG. 16A is an example plot of simulated RF channel data and estimated channel data from a speckle region of an ultrasound image of anechoic cysts according to an aspect of the present disclosure;

FIG. 16B is an example plot of simulated RF channel data and estimated channel data from a cyst region of an ultrasound image of anechoic cysts according to an aspect of the present disclosure;

FIG. 16C is an example plot of average cross-correlation coefficients for 64 channels according to an aspect of the present disclosure;

FIG. 17A is an example plot of average cross-correlation as a function of receive element spacing in a speckle region of an ultrasound image of anechoic cysts at 30 mm depth according to an aspect of the present disclosure;

FIG. 17B is an example plot of average cross-correlation as a function of receive element spacing in a cyst region of an ultrasound image of anechoic cysts at 30 mm depth according to an aspect of the present disclosure;

FIG. 17C is an example plot of average cross-correlation as a function of receive element spacing in a speckle region of an ultrasound image of anechoic cysts at 60 mm depth according to an aspect of the present disclosure;

FIG. 17D is an example plot of average cross-correlation as a function of receive element spacing in a cyst region of an ultrasound image of anechoic cysts at 60 mm depth according to an aspect of the present disclosure; and

FIG. 18 is a plot of probability density functions (PDF) of images made using DAS, SLSC, and LACE according to an aspect of the present disclosure.

### DETAILED DESCRIPTION

The ultrasound imaging systems, methods of using the same, and the computer readable medium described herein apply multiple filters to a beamformed signal to generate multiple filtered beamformed signals. The beamformed signal is a summation of channel signals, and the filtered beamformed signals reproduce the channel signals that were summed together. Beamforming may advantageously reduce data size of the channel signals and increase imaging efficiency. Normalized cross-correlation may be performed on multiple pairs of filtered beamformed signals to determine a coherence coefficient corresponding to each pixel of an ultrasound image, which may be used to construct a coherence estimation ultrasound image. Coherence is a measurement of similarity of echoes produced, and an image constructed based on coherence estimation may advantageously produce higher quality and higher contrast images of target regions, especially target regions that return a low amplitude echo (e.g., heart chamber, blood vessels near heart chamber). Coherence based systems produce images that show such target regions as black as opposed to clutters, and thus a user may better identify the regions and perform more accurate measurements on the images.

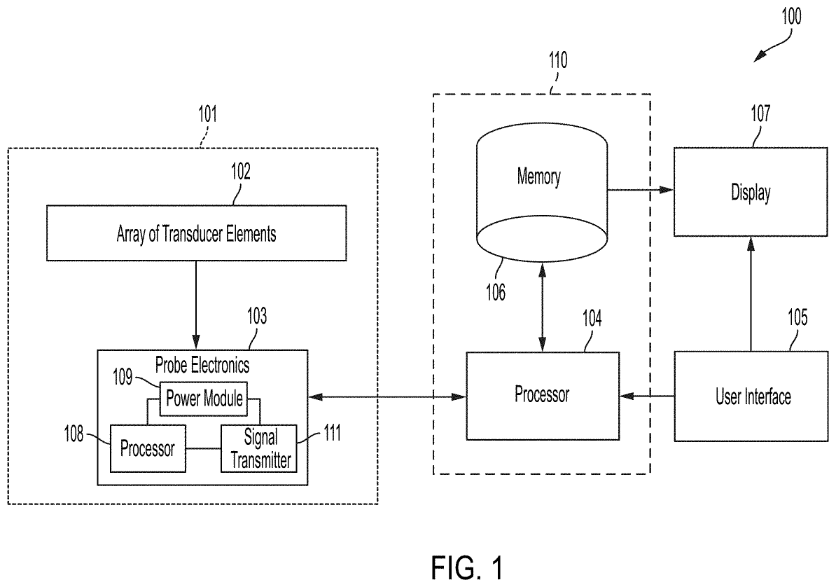

FIG. 1 is a schematic diagram of an example ultrasound imaging system 100 using coherence estimation of a beamformed signal and a UPS 110 for coherence estimation of a beamformed signal. The ultrasound imaging system 100 may include an ultrasound probe 101, a processor 104, a user interface 105, a memory 106, and a display 107. The ultrasound probe 101 may include an array of transducer elements 102 and probe electronics 103 that may be used to control the switching of the array of transducer elements 102. It should be appreciated that some embodiments may have arrays of transducer elements 102 with a varying number of transducer elements, and a variety of ways in which the transducer elements may be arranged. In some embodiments, the probe electronics 103 may include a processor 108, a power module 109, a signal transmitter 111, or some combination thereof. It should be appreciated that in some embodiments some or all of the probe electronics 103 may be physically located inside a probe casing (not shown) which may house some or all of the probe electronics 103 to protect the probe electronics 103 from the surrounding environment, while in other embodiments some or all of the probe electronics 103 may be physically located outside of a probe casing.

The processor 104 may include a single processor or multiple processors and may be configured to execute machine-readable instructions. The processor 104 may execute instructions to operate the array of transducers elements 102 to control an amount of power delivered to the array of transducer elements 102 and perform the coherence estimation and/or the reconstruction of the image. The processor 104 may be a microprocessor or a microcontroller by example.

The user interface 105 may be displayed on the display 107. The user interface 105 may be used to input parameters and control the ultrasound imaging system 100. By example and not limitation, an input may be received by the display 107 (i.e., touchscreen), buttons, keys, knobs, one or more cameras, or a microphone. The input may be touch, visual, and/or auditory. The received input may be biometric information, the user's voice, and/or the user's touch.

The memory 106 may be a non-transitory computer readable storage medium. For example, the memory 106 may be a random-access memory (RAM), a disk, a flash memory, optical disk drives, hybrid memory, or any other storage medium that can store data. The memory 106 may store program code that are executable by the processor 104. The memory 106 may store data in an encrypted or any other suitable secure form.

The ultrasound imaging system may include the UPS 110 or be coupled to the UPS 110 as shown in FIG. 1. The UPS 110 may include program instructions for performing the methods described herein. The UPS 110 may be executed by the one or more processors 104 of the ultrasound imaging system 100, alone or in combination with the probe electronics 103. The program instructions may be stored in the memory 106. The programming instructions may be implemented in C, C++, JAVA, or any other suitable programming language. In some embodiments, some or all of the portions of the UPS 110 including the subsystems or modules may be implemented in application specific circuitry such as Application Specific Integrated Circuits (ASICs) and Field Programmable Gate Arrays (FPGAs). In some implementations, some or all of the aspects of the functionality of the UPS 110 may be executed remotely on a server over a network.

In some embodiments, the UPS 110 may generate and/or beamform one or more signals that are transmitted towards a target by the array of transducer elements 102. In some embodiments, such beamforming may be performed using various beamforming methods known in the art, including but not limited to analog beamforming, digital beamforming, hybrid beamforming (part analog and part digital), Fresnel-based beamformer, minimum-variance beamformer, Capon beamformer, Wiener beamformer, and delay-and-multiply beamforming. The transmit beamforming applies appropriate time delays and weighings to an ultrasound signal for each transducer element in the array of transducer elements 102 in order to focus the transmitted ultrasound beam at the intended target.

In some embodiments, the UPS 110 may beamform a reflected signal received by the array of transducer elements 102. In some embodiments, the transmit and/or receive beamforming, or portions thereof, may be performed by components of the probe electronics 103, or other dedicated components of the ultrasound imaging system 100, such as an ASIC or an FPGA. In some embodiments, the UPS 110, by itself or in conjunction with the probe electronics 103, may be configured to populate an RF signal matrix based on the beamformed received signal.

The array of transducer elements 102 transmit beamformed ultrasonic signals into a target area or the tissue of a patient being examined. The ultrasonic signals reflect off structures in the body, like blood cells or muscular tissue, to produce echoes that return to the array of transducer elements 102. The echoes are converted into electrical signals, or RF signal data, by the transducer elements, and the received RF signal data is received by the processor 104. In some embodiments, the processor 104 assembles the received RF signal data into an RF signal matrix.

The UPS 110 may apply multiple spatial filters to the RF signal matrix. The spatial filters may be applied either in the time domain or the spatial frequency domain. The spatial frequency domain may also be referred to as k-space. To apply the spatial filters in k-space, a frequency transform (e.g., a 2D FFT) is applied to the RF matrix to produce the k-space representation of the RF matrix. The spatial filters are applied by multiplying the k-space representation of the RF matrix by the k-space representations of the multiple spatial filters, to produce k-space representations of multiple filtered RF matrices. An inverse frequency transform (e.g., a 2D Inverse Fast Fourier transform (2D IFFT)) is applied to the k-space representations of the multiple filtered RF matrices to produce multiple filtered RF matrices. In embodiments in which the spatial filters are applied in the time domain, convolution of the time domain representation of the RF matrix and the time domain representations of the multiple spatial filters is performed to produce multiple filtered RF matrices.

The multiple spatial filters may have varying degrees of overlap in k-space with each other. These varying degrees of overlap among the multiple spatial filters are used to estimate coherence of the received ultrasound wave. The UPS 110 may estimate such coherence by performing normalized cross-correlation of the k-space output of pairs of filtered data.

In some embodiments, the UPS 110 may perform the normalized cross-correlation on pairs of filtered RF matrices generated using spatial filters having between approximately 40 and 99.9% overlap with each other. In some embodiments, the UPS 110 may perform the normalized cross-correlation on pairs of filtered RF matrices with 80% overlap with each other. In some embodiments, the UPS 110 may perform the normalized cross-correlation on pairs of filtered RF matrices with 85% overlap with each other. In some embodiments, the UPS 110 may perform the normalized cross-correlation on pairs of filtered RF matrices with 90% overlap with each other. In some embodiments, the UPS 110 may perform the normalized cross-correlation on pairs of filtered RF matrices with 95% overlap with each other.

In some embodiments, the UPS 110 may perform the normalized cross-correlation on between 10 and 100 pairs of filtered RF matrices. In some embodiments, the UPS 110 may perform the normalized cross-correlation on less than 10 pairs of filtered RF matrices. In some embodiments, the UPS 110 may perform the normalized cross-correlation on more than 100 pairs of filtered RF matrices.

By applying the spatial filters to the beamformed signal, instead of processing channel data from individual transducer elements as is done in other coherence estimation techniques, embodiments of the improved ultrasound imaging system described herein are able to improve the contrast-to-noise and signal-to-noise ratios of a standard ultrasound image without requiring the system to process large amounts of data in real time. For example, a 64-channel beamformer with 12-bit A/Ds running at 40 MHz, requires transferring data from the probe at a rate of 3.58 gigabytes/second in order to perform coherence calculations on individual channel data. In comparison, an embodiment of the improved ultrasound imaging system 100 described herein, using beamformed data to perform coherence calculations on the received signal, may only require transferring data from the probe at a rate of 57.2 megabytes/second, while still providing improved contrast-to-noise and signal-to-noise ratios of a standard ultrasound image. Such improved image quality may also permit the use of the array of transducer elements 102 with a lower number of elements than would otherwise be required for a desired image quality.

In some embodiments, the user interface 105 may be used to receive user input to control operation of the ultrasound imaging system 100, including to control various parameters of ultrasound imaging system, such as imaging start depth, imaging end depth, number of lines, line spacing, and/or sampling frequency, and to control various parameters of the display 107, such as gain and/or contrast of a displayed image.

In some embodiments, the UPS 110 may process the received RF data and prepare ultrasound images for display on the display 107. In some embodiments, processing of the received RF data to prepare an ultrasound image may include applying multiple spatial filters to the received RF data to generate filtered RF data. Processing of the received RF data to prepare an ultrasound image may include performing normalized cross-correlation on the filtered RF data to determine a coherence coefficient corresponding to each pixel of an ultrasound image from which a coherence estimation ultrasound image using coherence estimation is constructed. In some embodiments, the multiple spatial filters may be stored on memory 106. In some embodiments, the prepared ultrasound images may be stored on memory 106 prior to being displayed on display 107. The memory 106 may comprise any known data storage medium.

Some embodiments of the improved ultrasound imaging system 100 described herein may include multiple processors to handle the processing tasks. For example, a first processor may be utilized to demodulate and decimate the ultrasound signal while a second processor may be used to further process the data prior to displaying an image. It should be appreciated that other embodiments may use a different arrangement of processors.

In some embodiments of the improved ultrasound imaging system 100 described herein, ultrasound signals may be processed in various ways according to program instructions, including beamforming, de-noising, and filtering ultrasound signals, or any portion or combination thereof. The UPS 110 may include program instructions that may be implemented on one or more processors 104, alone or in combination with the probe electronics 103. The program instructions may be stored on memory 106 of the system. In some embodiments, the program instructions correspond to the processes and functions described herein, and may be executed by a processor, such as the processor 104. In some embodiments, the program instructions may be implemented in C, C++, JAVA, or any other suitable programming language. In some embodiments, some or all of the portions of the program instructions may be implemented in application specific circuitry including ASICs and FPGAs, and such application specific circuitry may be part of the probe electronics 103, or another part of the ultrasound imaging system 100.

For example, in an embodiment, programming instructions may be provided to generate B-mode image frames and corresponding RF matrices based on a reflected and received ultrasound signal, spatial filters and corresponding filtered RF matrices, and final improved coherence estimation image frames based on the filtered RF matrices. The image frames may be stored along with timing information indicating a time at which the image frame was acquired in the memory 106 may be recorded with each image frame. Programming instructions may be provided to retrieve stored image frames from the memory 106 and to display the image frames on the display 107.

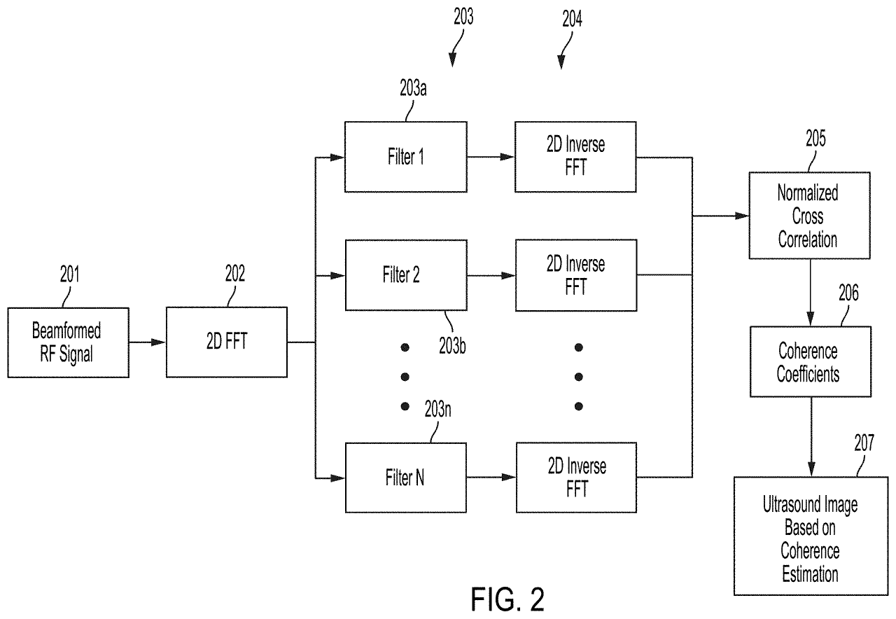

FIG. 2 show a process flow diagram for an embodiment of a method of constructing an improved ultrasound imaging using coherence estimation of a beamformed signal that may be performed by the UPS 110 (see FIG. 1). Signals may be beamformed using any method known in the art, including but not limited to analog beamforming, digital beamforming, hybrid beamforming (part analog, part digital), Fresnel-based beamformer, minimum-variance beamformer, Capon beamformer, Wiener beamformer, and delay-and-multiply beamforming. First, the UPS 110 may transform a beamformed RF ultrasound signal 201, which may be represented in the form of an RF signal matrix, into k-space using frequency transform. In an embodiment, the frequency transform may be a 2D FFT 202 to generate a k-space representation of the RF signal. In other embodiments, the frequency transform may be a fast Fourier transform, discrete Fourier transform, cosine transform, discrete cosine transform, sine transform, discrete sine transform, wavelet transform, discrete wavelet transform, short time Fourier transform, discrete short time Fourier transform, Laplace transform, discrete Laplace transform, fractional Fourier Transform, discrete fractional Fourier transform, and 3D frequency transforms such as 3D fast Fourier transform, and 3D discrete Fourier transform.

The UPS 110 (see FIG. 1) may apply multiple spatial filters 203a-n (where n is the total number of spatial filters for a given embodiment) to the k-space representation of the RF signal to generate k-space representations of multiple filtered RF signals. In some embodiments, the spatial filters may be represented as matrices with dimensions such that the filters are applied by multiplying k-space representation of each filter by the k-space representation of the RF signal matrix to generate multiple k-space representations of filtered RF signal matrices.

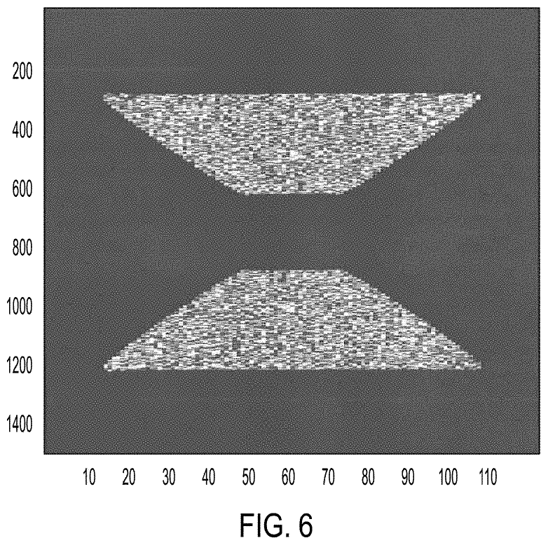

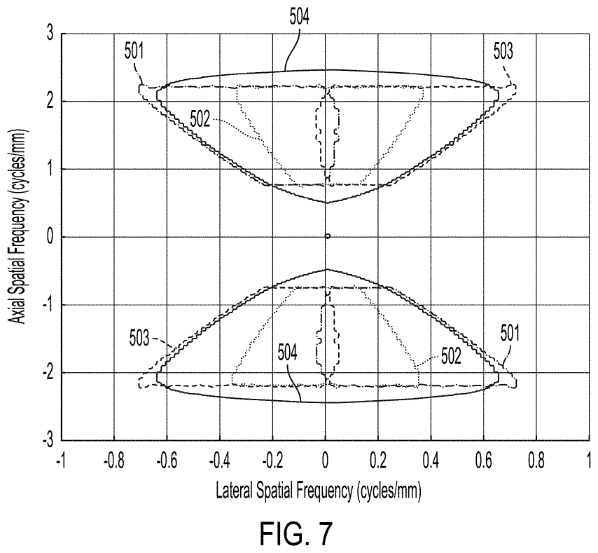

In some embodiments, the spatial filters may be represented as matrices. In some embodiments, the matrices may be populated with values equal to 0 or 1, or any value in between. In some embodiments, spatial filters could be complex-valued, having real and imaginary components. In some embodiments, each of the multiple spatial filters may be randomly or pseudo-randomly generated. In some embodiments, each of the multiple spatial filters may be randomly or pseudo-randomly generated such that a certain predetermined percentage of the entries in a given filter matrix are equal to 0 and a certain predetermined percentage of the entries in a given filter matrix are equal to 1. For example, in an embodiment, each of the multiple matrices may have 80% of its entries equal to 1, and 20% of its entries equal to 0, with the distribution of such entries within the matrix randomized or pseudo-randomized. In such an example, any two of the multiple filters would be expected to have approximately 64% overlap with one another. In some embodiments, the multiple spatial filters are generated such that the cumulative coverage of the spatial filters in k-space overlaps with all or most of the k-space representation of the ultrasound RF data. In some embodiments, the multiple spatial filters may be generated such that each spatial filter has at least approximately 40% overlap with adjacent spatial filter(s). In some embodiments, spatial filters may overlap with adjacent spatial filters in the axial direction, the lateral direction, or a combination thereof. In some embodiments, the multiple spatial filters may be generated such that each spatial filter has between approximately 40% and approximately 99.9% overlap with at least one other spatial filter. Certain embodiments of k-space representations of spatial filters are shown in FIGS. 6 and 7.

The UPS 110 (see FIG. 1) may generate spatial filters prior to imaging and store the spatial filters in computer memory accessible by the ultrasound imaging system, or the spatial filters may be generated during the imaging process, or a combination thereof. In some embodiments, between approximately 10 and 100 spatial filters may be used. In some embodiments, less than 10 spatial filters may be used. In some embodiments, more than 100 spatial filters may be used.

Next, the UPS 110 (see FIG. 1) may transform the multiple k-space representations of filtered RF signal matrices using an inverse frequency transform to generate multiple filtered RF signal matrices. In one embodiment, the inverse frequency transform is a 2D IFFT 204. In other embodiments, the inverse frequency transform may be an inverse fast Fourier transform, inverse discrete Fourier transform, inverse cosine transform, inverse discrete cosine transform, inverse sine transform, inverse discrete sine transform, inverse wavelet transform, inverse discrete wavelet transform, inverse short time Fourier transform, inverse discrete short time Fourier transform, inverse Laplace transform, inverse discrete Laplace transform, inverse fractional Fourier transform, discrete inverse fractional Fourier transform, and 3D inverse frequency transforms such as 3D inverse fast Fourier transform, and 3D inverse discrete Fourier transform.

Next, the UPS 110 (see FIG. 1) may perform normalized cross-correlation 205 between multiple pairs of filtered RF signals to determine a cross-correlation coefficient for each pair of filtered RF signals. In some embodiments, signal similarity may be measured using other techniques known in the art, including cross-correlation (without normalization), sum of absolute differences, n-bit correlators such as a 2-bit correlator, sign comparator, and triple correlation. In some embodiments, the pairs of filtered RF signals may be represented as pairs of filtered RF signal matrices. In some embodiments, the normalized cross-correlation is performed on corresponding segments of data approximately 1-4 wavelengths long in the axial direction from each filtered RF signal in a pair. In some embodiments, the UPS 110 may perform the normalized cross-correlation between two sets of filtered RF signal matrices on segments of data less than 1 wavelength long in the axial direction or more than 4 wavelengths long in the axial direction.

In some embodiments, the UPS 110 (see FIG. 1) may perform normalized cross-correlation on each pair of filtered RF signals for which the corresponding spatial filters have between approximately 40% and approximately 99.9% overlap with each other. In some embodiments, the UPS 110 may perform normalized cross-correlation on pairs of filtered RF signals for which the corresponding spatial filters have approximately 80% overlap. In some embodiments, the UPS 110 may perform normalized cross-correlation on pairs of filtered RF signals for which the corresponding spatial filters have approximately 85% overlap with each other. In some embodiments, the UPS 110 may perform normalized cross-correlation on pairs of filtered RF signals for which the corresponding spatial filters have approximately 90% overlap with each other. In some embodiments, the UPS 110 may perform normalized cross-correlation on pairs of filtered RF signals for which the corresponding spatial filters have approximately 95% overlap with each other. In some embodiments, the UPS 110 may perform normalized cross-correlation on pairs of filtered RF signals for which the corresponding spatial filters have less than 40% overlap. In some embodiments, the UPS 110 may perform normalized cross-correlation on pairs of filtered RF signals for which the corresponding spatial filters have more than 95% overlap.

In some embodiments, the correlation between two signals may be quantified using the following equation, where sᵢ is the time domain signal from filter i, and sⱼ is the time domain signal from filter j:

$${\hat{C}}_{i j} ( t ) = {\sum }_{τ = - T / 2}^{T / 2} {s}_{i} ( t + τ ) {s}_{j} ( t + τ )$$

In some embodiments, the coherence between two signals may be quantified using the normalized cross-correlation:

$${\hat{R}}_{i j} ( t ) = \frac{{\hat{C}}_{i j} ( t )}{\sqrt{{\hat{C}}_{i j} ( t ) {\hat{C}}_{j j} ( t )}}$$

In some embodiments, a coherence estimate may be performed using the following equation, where ξ is the set of signals that are cross-correlated with signal i:

$${\hat{R}}_{p , LACE} ( t ) = {\sum }_{i = 1}^{N} {\sum }_{j ∈ ξ} {\hat{R}}_{ij} ( t )$$

The cross-correlation coefficients for each pair of filtered RF signals are used to determine a coherence coefficient 206 corresponding to each pixel of a coherence estimation ultrasound image. In some embodiments, the cross-correlation coefficients are scan converted. In some embodiments, the cross-correlation coefficients relating to a given pixel are summed to determine a coherence coefficient for that pixel. In some embodiments, the cross-correlation coefficients relating to a given pixel are weighed and then summed to determine a coherence coefficient for that pixel. In other embodiments, the cross-correlation coefficients relating to a given pixel are averaged to determine a coherence coefficient for that pixel.

The coherence coefficients 206, each corresponding to a pixel of an ultrasound image, are used to generate a coherence estimation ultrasound image based on coherence estimation 207. In some embodiments, the coherence coefficients 206 are used to generate a coherence estimation ultrasound image based on coherence estimation using a grayscale conversion in which a coherence coefficient of 0 is mapped to total black, and a coherence coefficient of 1 is mapped to total white. The grayscale may be linear or non-linear. In some embodiments, the coherence estimation ultrasound image based on coherence estimation 207 may be displayed on a screen for visualization by a user, such as a physician or an ultrasound technician, depending on the application.

By applying the spatial filters to the beamformed signal, instead of processing channel data from individual transducer elements as is done in other coherence estimation techniques, embodiments of the improved ultrasound imaging systems described herein may be able to improve the contrast-to-noise and signal-to noise ratios of a standard ultrasound image without requiring the system to process large amounts of data in real time. For example, a 64-channel beamformer with 12-bit A/Ds running at 40 MHz, requires transferring data from the probe at a rate of 3.58 gigabytes/second in order to perform coherence calculations on individual channel data. In comparison, an embodiment of the improved ultrasound imaging system described herein, using beamformed data to perform coherence calculations on the received signal, may only require transferring data from the probe at a rate of 57.2 megabytes/second, while still providing improved contrast-to-noise and signal-to-noise ratios of a standard ultrasound image.

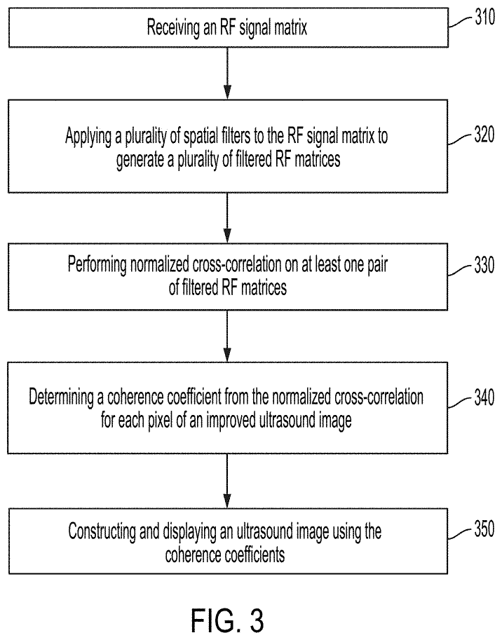

FIG. 3 shows a process flow diagram for an embodiment of a method of improved ultrasound imaging using coherence estimation that may be performed by the UPS 110 (see FIG. 1). In block 310, the UPS 110 receives an RF signal matrix. The RF signal matrix may be an array of RF signals coming from multiple channels of a digitizer. Said differently, the RF signal matrix may be combined ultrasound channel data of multiple channels.

In block 320, the UPS 110 (see FIG. 1) applies multiple spatial filters to the RF signal matrix to generate multiple filtered RF signal matrices. The UPS 110 may apply the spatial filters in the time domain or in k-space. To apply a spatial filter in k-space, the UPS 110 applies a frequency transform to the RF signal to the RF signal matrix to produce a k-space representation of the RF signal matrix. In some embodiments, the frequency transform may be a 2D FFT. The UPS 110 may then apply the multiple spatial filters by multiplying the k-space representation of the RF signal matrix by the k-space representations of the multiple spatial filters to produce k-space representations of multiple filtered RF signal matrices. The UPS 110 may then apply an inverse frequency transform to the multiple filtered RF signal matrices to produce multiple filtered RF signal matrices. In some embodiments, the inverse frequency transform may be a 2D IFFT.

In some embodiments, the spatial filters may be represented as matrices with dimensions such that the filters are applied by multiplying k-space representation of each filter by the k-space representation of the RF signal matrix to generate multiple k-space representations of filtered RF signal matrices.

In some embodiments, the spatial filters may be represented as matrices. In some embodiments, the matrices may be populated with values equal to 0 or 1, or any value in between. In some embodiments, each of the multiple spatial filters may be randomly or pseudo-randomly generated. In some embodiments, each of the multiple spatial filters may be randomly or pseudo-randomly generated such that a certain predetermined percentage of the entries in a given filter matrix are equal to 0 and a certain predetermined percentage of the entries in a given filter matrix are equal to 1. For example, in an embodiment, each of the multiple matrices may have 80% of its entries equal to 1, and 20% of its entries equal to 0, with the distribution of such entries within the matrix randomized or pseudo-randomized. In such an example, any two of the multiple filters would be expected to have approximately 64% overlap with one another. In some embodiments, the multiple spatial filters are generated such that the cumulative coverage of the spatial filters in k-space overlaps with all or most of the k-space representation of the ultrasound RF data. In some embodiments, the multiple spatial filters may be generated such that each spatial filter has at least approximately 40% overlap with adjacent spatial filter(s). In some embodiments, spatial filters may overlap with adjacent spatial filters in the axial direction, the lateral direction, or a combination thereof. In some embodiments, the multiple spatial filters may be generated such that each spatial filter has between approximately 40% and approximately 99.9% overlap with at least one other spatial filter. Certain embodiments of k-space representations of spatial filters are shown in FIGS. 6 and 7.

The spatial filters may be generated prior to imaging and stored in computer memory accessible by the ultrasound imaging system, or the spatial filters may be generated during the imaging process, or a combination thereof. In some embodiments, between approximately 10 and 100 spatial filters may be used. In some embodiments, less than 10 spatial filters may be used. In some embodiments, more than 100 spatial filters may be used.

By applying the spatial filters to the beamformed signal, instead of processing channel data from individual transducer elements as is done in other coherence estimation techniques, embodiments of the improved ultrasound imaging methods described herein may be able to improve the contrast-to-noise and signal-to-noise ratios of a standard ultrasound image without requiring the system to process large amounts of data in real time. For example, a 64-channel beamformer with 12-bit A/Ds running at 40 MHz requires transferring data from the probe at a rate of 3.58 gigabytes/second in order to perform coherence calculations on individual channel data. In comparison, an embodiment of the improved ultrasound imaging system described herein, using beamformed data to perform coherence calculations on the received signal, may only require transferring data from the probe at a rate of 57.2 megabytes/second, while still providing improved contrast-to-noise and signal-to-noise ratios of a standard ultrasound image.

In block 330, the UPS 110 (see FIG. 1) performs normalized cross-correlation on multiple pairs of filtered RF signal matrices. In some embodiments, the UPS 110 may perform the normalized cross-correlation on corresponding segments of data approximately 1-4 wavelengths long in the axial direction from each filtered RF signal matrix in a pair. In some embodiments, the UPS 110 may perform the normalized cross-correlation between two sets of filtered RF signal matrices on segments of data less than 1 wavelength long in the axial direction or more than 4 wavelengths long in the axial direction.

In some embodiments, the UPS 110 (see FIG. 1) may perform the cross-correlation on each pair of filtered RF signal matrices for which the corresponding spatial filters have between approximately 40% and approximately 99.9% overlap. In some embodiments, the UPS 110 may perform the normalized cross-correlation on pairs of filtered RF signal matrices for which the corresponding spatial filters have approximately 80% overlap. In some embodiments, the UPS 110 may perform the normalized cross-correlation on pairs of filtered RF signal matrices for which the corresponding spatial filters have less than 40% overlap. In some embodiments, the UPS 110 may perform the normalized cross-correlation on pairs of filtered RF signal matrices for which the corresponding spatial filters have more than 95% overlap.

In some embodiments, the correlation between two signals may be quantified using the following equation, where sᵢ is the time domain signal from filter i, and sⱼ is the time domain signal from filter j:

$${\hat{C}}_{i j} ( t ) = {\sum }_{τ = - T / 2}^{T / 2} {s}_{i} ( t + τ ) {s}_{j} ( t + τ )$$

In some embodiments, the coherence between two signals may be quantified using the following normalized cross-correlation:

$${\hat{R}}_{i j} ( t ) = \frac{{\hat{C}}_{i j} ( t )}{\sqrt{{\hat{C}}_{i j} ( t ) {\hat{C}}_{j j} ( t )}}$$

In some embodiments, a coherence estimate may be performed using the following equation, where is the set of signals that are cross-correlated with signal i:

$${\hat{R}}_{p , LACE} ( t ) = {\sum }_{i = 1}^{N} {\sum }_{j ∈ ξ} {\hat{R}}_{ij} ( t )$$

In block 340, the UPS 110 (see FIG. 1) uses the cross-correlation coefficients for each pair of filtered RF signal matrices to determine a coherence coefficient corresponding to each pixel of a coherence estimation ultrasound image. In some embodiments, the UPS 110 may sum the cross-correlation coefficients relating to a given pixel to determine a coherence coefficient for that pixel. In some embodiments, the UPS 110 may weigh and then sum the cross-correlation coefficients relating to a given pixel to determine a coherence coefficient for that pixel. In other embodiments, the UPS 110 may average the cross-correlation coefficients relating to a given pixel to determine a coherence coefficient for that pixel. In some embodiments, the UPS 110 may use only a subset of the cross-correlation coefficients relating to a given pixel to determine the coherence coefficient for that pixel.

In block 350, the UPS 110 (see FIG. 1) uses the coherence coefficients each corresponding to a pixel of an ultrasound image to construct a coherence estimation ultrasound image. In some embodiments, the UPS 110 uses a grayscale conversion, in which a coherence coefficient of 0 is mapped to total black and a coherence coefficient of 1 is mapped to total white, to construct a coherence estimation ultrasound image. The grayscale may be linear or non-linear. In some embodiments, the UPS 110 may display the coherence estimation ultrasound image on a screen for visualization by a user, such as a physician or ultrasound technician, depending on the application. In some embodiments, the coherence estimation ultrasound image is improved over prior art ultrasound images with higher contrast-to-noise and/or signal-to-noise ratios than ultrasound images processed without using the coherence estimation methods described herein.

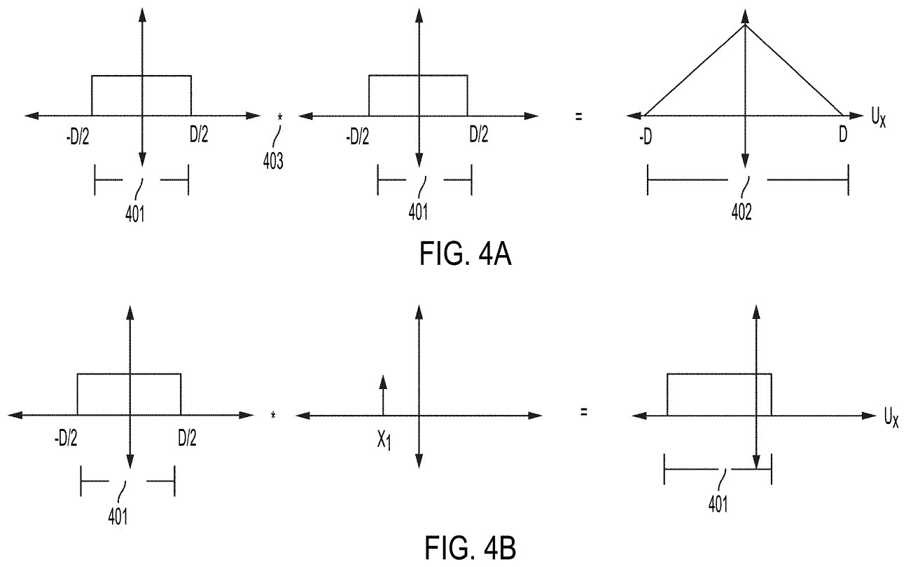

FIG. 4A illustrates a transmit-receive convolutional process with an aperture width 401 of D yielding a lateral k-space function having a triangle function with a width 402 of 2D. An aperture width 401 of D may be assumed to be used in both transmit and receive. The aperture may be uniformly weighted. If a Fourier Transform relationship between the aperture and a point spread function at a focal point is assumed, transmit and receive point spread functions may each be

$$rect ( \frac{{x}_{0}}{D} ) .$$

Additionally, x₀ may De the lateral aperture coordinate, x may be the lateral beam coordinate, X may be the ultrasound wavelength, and z may be the focal depth. The double-sided arrow may indicate that the two expressions are Fourier Transform pairs. The transmit-receive convolutional process may be represented by the below expression.

$$rect ( \frac{{x}_{0}}{D} ) ↔ sin c ( \frac{π D x}{λ z} )$$

Due to the multiplicative process of transmit and receive and assuming the same aperture is used in transmit and receive, the transmit-receive point spread function may be as shown below.

$$rect ( \frac{{x}_{0}}{D} ) * rec t ( \frac{{x}_{0}}{D} ) ↔ sin {c}^{2} ( \frac{π D x}{λ z} )$$

A Fourier Transform may be performed to obtain the frequency representation of the transmit-receive point spread function indicated by F{ } on the transmit-receive point spread function to yield the below equation.

$${F}_{s y s} ( {u}_{x} ) = F \{ sin {c}^{2} ( \frac{π D x}{λ z} ) \} = tr i ( \frac{λ z}{π D} {u}_{x} )$$

In the above expression, tri may be a triangle function defined as

$$t r i ( x ) = ( 1 - \vert x \vert ) rect ( \frac{x}{2} )$$

and uₓ may be the lateral spatial frequency. The frequency domain representation of the point spread function may be also referred to as a k-space representation or a transfer function of the ultrasound imaging system 100. Notably, the width of the triangle function may be proportional to twice the width of the aperture D. The same triangle function may be arrived at through convolution of the two rectangular apertures each having a width 401 of D because of the Fourier Transform relationship between the aperture and the point spread function. Asterisk 403 of FIG. 4A may indicate convolution.

FIG. 4B illustrates a transmit-receive convolutional process with a transmit aperture width 401 of D and a receive aperture of an element approximated as a delta function yielding a lateral k-space function that is a rectangle function of width 401 of D centered at x₁. The aperture width 401 of D may be used in transmit and a single receive element located at x₀=x₁ is used where the receive element is located anywhere between −D/2 and +D/2. The receive element may be approximated by a delta function δ(x₀−x₁) due to the small width of the receive element. The transmit receive point spread function may be a sinc function multiplied by a linear phase tilt

$$e \frac{- j k x {x}_{1}}{z} ,$$

and its k-space representation may be a rectangle function of width 401 of D centered at a location corresponding to the element position. This process may be expressed by the below equation.

$$rect ( \frac{{x}_{0}}{D} ) * δ ( {x}_{0} - {x}_{1} ) = r e c t ( \frac{{x}_{0} - {x}_{1}}{D} ) ↔ sin c ( \frac{π D x}{λ z} ) {e}^{\frac{- j k x {x}_{1}}{λ z}}$$

Based on the processes shown in FIGS. 4A-4B, the k-space coverage using a single receive element may be a portion or subset of the k-space coverage when using the entire receive aperture. Thus, the beamformed RF signal may be filtered from the entire receive aperture to produce an estimate of the signal from a receive element located at x₁.

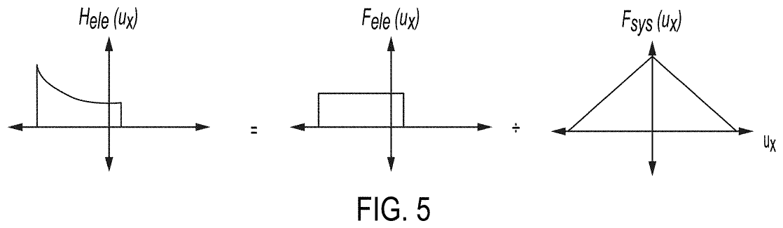

FIG. 5 illustrates obtaining an estimate of an element response in k-space by dividing a single element response by an overall k-space response of a full aperture. In the frequency domain a filter function, Hₑₗₑ(uₓ), may be created by taking the frequency response of a single element, Fₑₗₑ(uₓ), and dividing by the frequency response when the entire aperture, F_{sys}(uₓ), may be used in both transmit and receive modes as shown by the below equation.

$${H}_{ele} ( {u}_{x} ) = \frac{{F}_{ele} ( {u}_{x} )}{{F}_{s y s} ( {u}_{x} )}$$

After Hₑₗₑ(uₓ) has been obtained for all elements, estimates of channel data may be obtained for any imaging target when F_{sys}(uₓ) is replaced with the 2D DFT of the beamformed RF matrix. The estimated channel data may be obtained by multiplying the 2D DFT of the beamformed RF matrix with Hₑₗₑ(uₓ) and then takin the inverse 2D DFT. Coherence estimation may be performed using the estimated channel data in same manner as SLSC.

After filtering, a 2D inverse DFT may be performed to obtain the filter outputs in the space/time domain. The filter outputs may be intended to provide estimates of the RF channel data. Having varying degrees of overlap among the filters and performing normalized cross-correlation of the filter output data may be used to approximate the coherence of the received ultrasound wave.

If the cross-correlation coefficient is high, signals may be estimated to be highly coherent. If the cross-correlation coefficient is low, signals may be estimated to have low coherence. If several dozens of filters are used, many combinations of pairs of filtered data may be used to produce many cross-correlation coefficients for a single pixel. The coefficients may then be summed to produce a final pixel value in the coherence image using the below equation.

$$\hat{R} ( m ) = \frac{1}{N - m} {\sum }_{i = 1}^{N - m} \frac{{\sum }_{n = {n}_{1}}^{{n}_{2}} {s}_{i} ( n ) {s}_{i + m} ( n )}{\sqrt{{\sum }_{n = {n}_{1}}^{{n}_{2}} {s}_{i}^{2} ( n ) {\sum }_{n = {n}_{1}}^{{n}_{2}} {s}_{i + m}^{2} ( n )}}$$

FIG. 6 shows an embodiment of a k-space representation of a spatial filter. In the embodiment shown, the values within the region of the spatial filter that do not overlap with the k-space representation of the RF signal are set to 0 and displayed as black, while the values within the all or part of regions of the spatial filter that overlap with the k-space representation of the RF signal are randomly or pseudo-randomly generated as 1 or 0. In the embodiment shown in FIG. 6, there are two such regions of randomly or pseudo-randomly generated values, generally in the shape of mirror image trapezoids as viewed in k-space, covering roughly the same area as the k-space representation of a beamformed ultrasound signal.

FIG. 7 shows one embodiment of the k-space representations of a set of three spatial filters 501, 502, and 503, plotted on a single k-space plot, each spatial filter having at least some overlap in the axial and lateral direction with the other two spatial filters. Spatial filter 501 may be obtained from k-space coverage using left elements. Spatial filter 502 may be obtained from k-space coverage using middle elements. Spatial filter 503 may be obtained from k-space coverage using right elements. Each of the spatial filters is represented by the boundary of an active portion of each filter and an inactive portion of each filter. For example, in one embodiment, values within the boundary of a given spatial filter may be set to 1, and values outside the boundary may be set to 0. In another embodiment, values within the boundary may be randomized or pseudo-randomized (e.g., randomized or pseudo-randomized to a value between 0 and 1), and values outside the boundary may be set to 0. Embodiments with three spatial filters, such as the embodiment shown in FIG. 7, may produce three pairs of filtered RF matrices: the pairs of spatial filters 501, 502, spatial filters 501, 503, and spatial filters 502, 503.

The contour 504 may indicate k-space coverage when using all 64 elements in transmit and receive. The k-space coverage may be of a 2.5 MHz, 64-element phased array with 50%-6 dB fractional bandwidth. A contour of the magnitude of the Fourier Transform may appear similar to the contour 504.

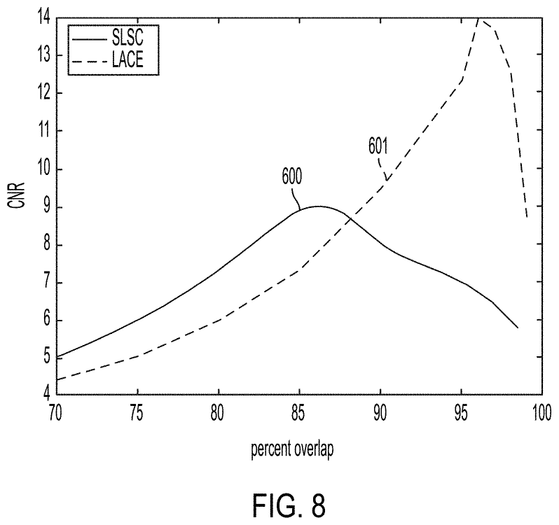

FIG. 8 is a plot of the CNR ratios achieved by the SLSC technique and the LACE technique as a function of the percentage of overlap of filters used for each technique, modeled using the Field II simulation program. For the embodiments of coherence estimation used to generate the data for FIG. 8, spatial filters were randomly or pseudo-randomly generated such that the average overlap between any given pair of filters could be calculated. The CNR ratio achieved by the SLSC technique as a function of the percentage of overlap of filters is shown as a solid line 600. The CNR ratio achieved by the LACE technique as a function of the percentage of overlap of filters is shown as a dashed line 601.

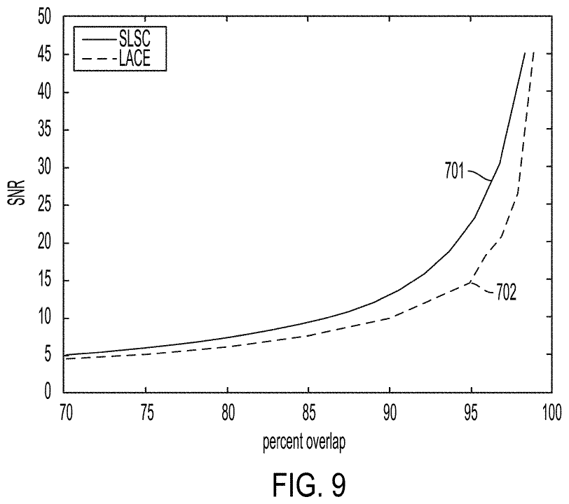

FIG. 9 is a plot of the SNR ratios achieved by the SLSC technique and the LACE technique as a function of the percentage of overlap of filters used for each technique, as modeled using the Field II simulation program. For the embodiments of coherence estimation used to generate the data for FIG. 9, spatial filters were randomly or pseudo-randomly generated such that the average overlap between any given pair of filters could be determined. The SNR ratio achieved by the SLSC technique as a function of the percentage of overlap of filters is shown as a solid line 701. The SNR ratio achieved by the LACE technique as a function of the percentage of overlap of filters is shown as a dashed line 702.

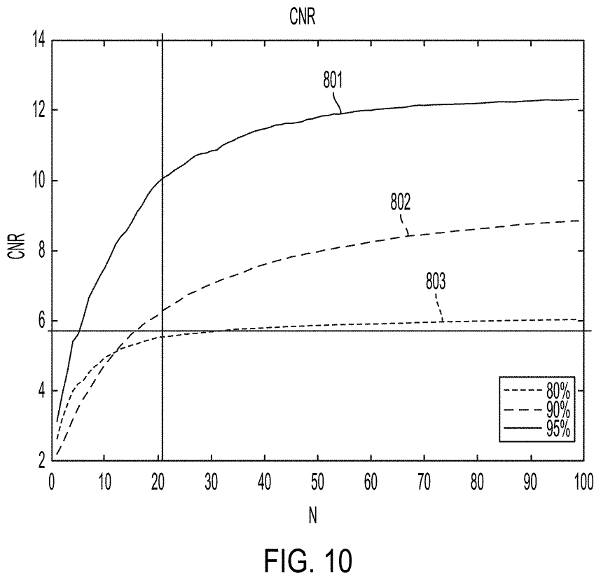

FIG. 10 is a plot of the CNR for embodiments of the coherence estimation methods described herein as a function of N, as modeled using the Field II simulation program. Spatial filters were randomly or pseudo-randomly generated such that the average overlap between any given pair of filters was 80%, 90%, and 95% respectively for each of the three lines in the plot. The average overlap of 95% is shown as a solid line 801, 90% is shown as a dashed line 802, and 80% is shown as a dotted line 803. The cross-hatch may represent the optimal combination of CNR and SNR achievable using the SLSC technique, as modeled using the Field II simulation program.

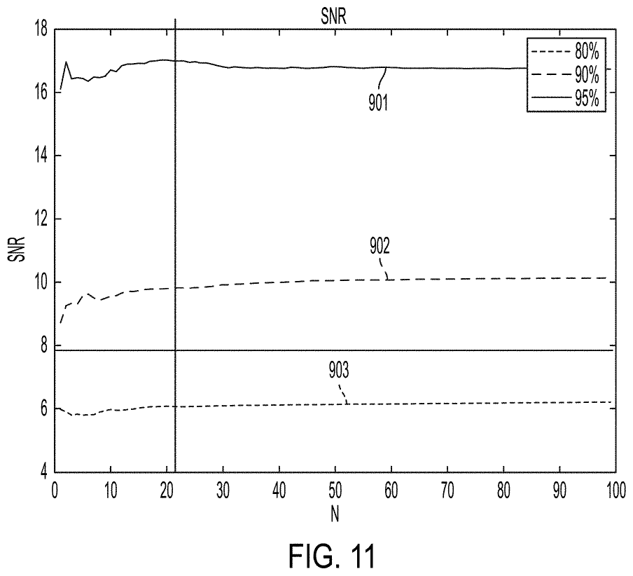

FIG. 11 is a plot of the SNR for embodiments of the coherence estimation methods described herein as a function of N, as modeled using the Field II simulation program. Spatial filters were randomly or pseudo-randomly generated such that the average overlap between any given pair of filters was 80%, 90%, and 95% respectively for each of the three lines in the plot. The average overlap of 95% is shown as a solid line 901, 90% is shown as a dashed line 902, and 80% is shown as a dotted line 903. The cross-hatch represents the optimal combination of CNR and SNR achievable using the SLSC technique, as modeled using the Field II simulation program.

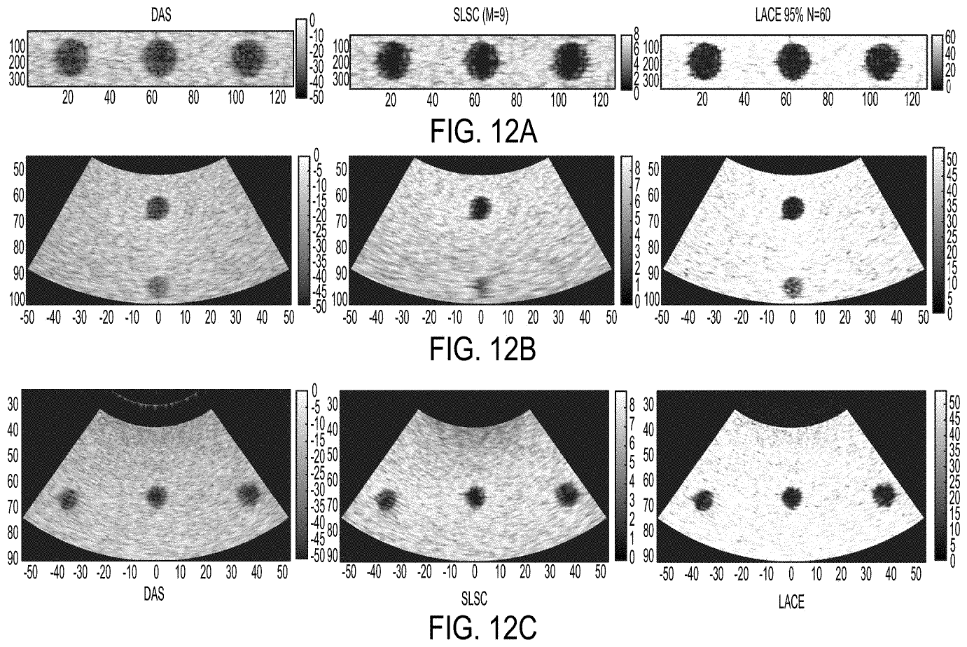

FIGS. 12A-12C are sets of ultrasound images generated using the Field II simulation program of DAS, SLSC, and LACE. The images are of anechoic cysts using standard DAS beamforming, SLSC, and LACE. The LACE embodiment uses 60 filters with an average overlap of 95% between pairs of filters. Table 1 shows CNR and SNR values for different anechoic cyst images shown in FIGS. 12A-12C using DAS, SLSC, and LACE.

TABLE 1

| | CNR | | | SNR | | |
|---|---|---|---|---|---|---|
| Figure | DAS | SLSC | LACE | DAS | SLSC | LACE |
| 12 A | 3.82 | 5.86 | 11.96 | 2.08 | 7.81 | 16.79 |
| 12 B | 3.81 | 4.47 | 9.33 | 2.50 | 8.82 | 13.38 |
| 12 C | 3.75 | 5.20 | 10.65 | 2.53 | 7.99 | 14.52 |

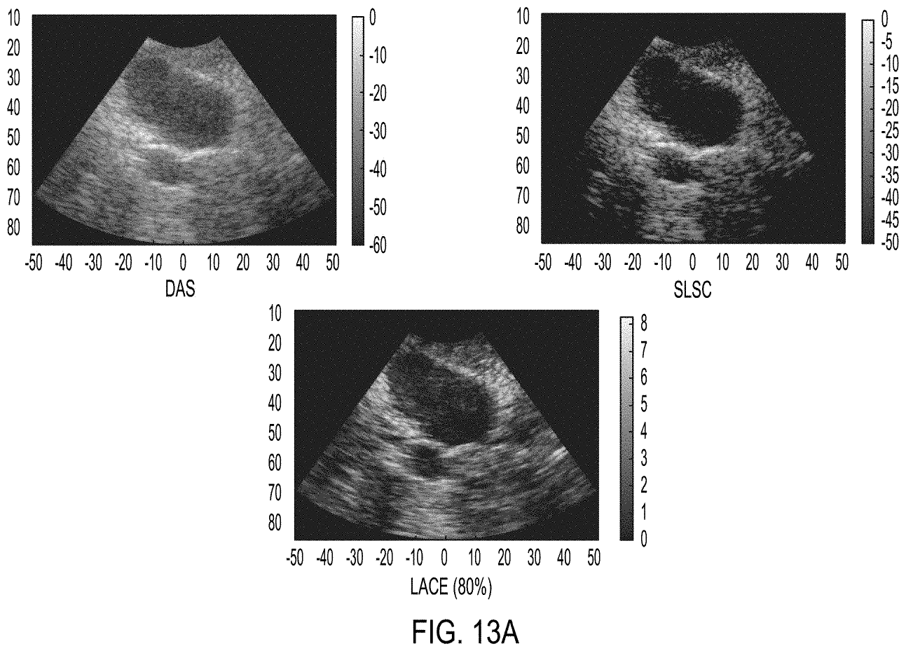

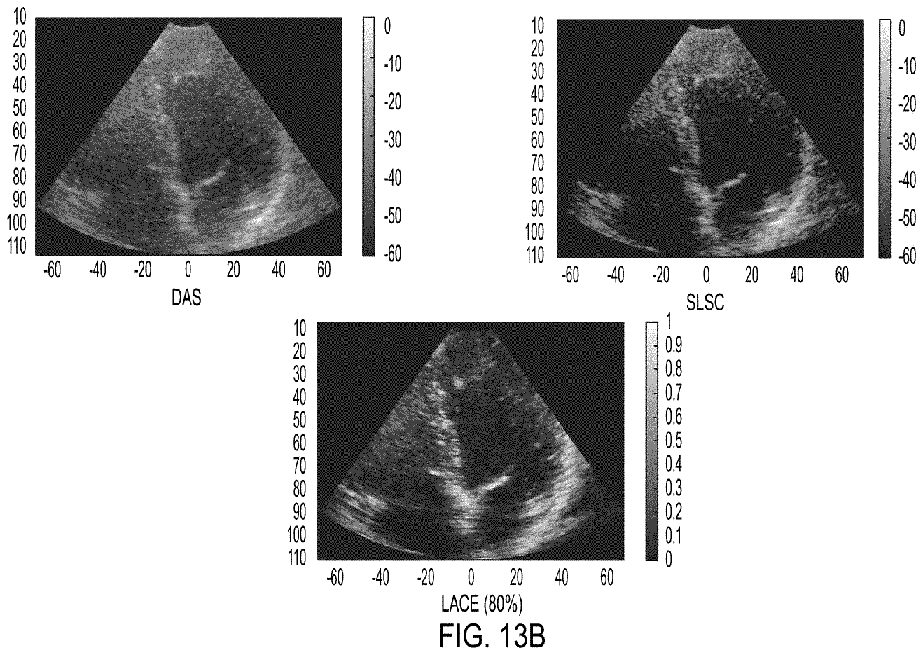

FIG. 13A shows a series of in vivo images of a gall bladder processed using traditional DAS, traditional SLSC, and LACE with 80% overlap. The in vivo images of the gall bladder may be in long axis and short axis. FIG. 13B shows a series of in vivo images of a heart in the 4-chamber apical view processed using traditional DAS, traditional SLSC, and LACE with 80% overlap. CNR and SNR values may be calculated by taking a part of the left atrium as the background and the central portion of the left ventricle as the target.

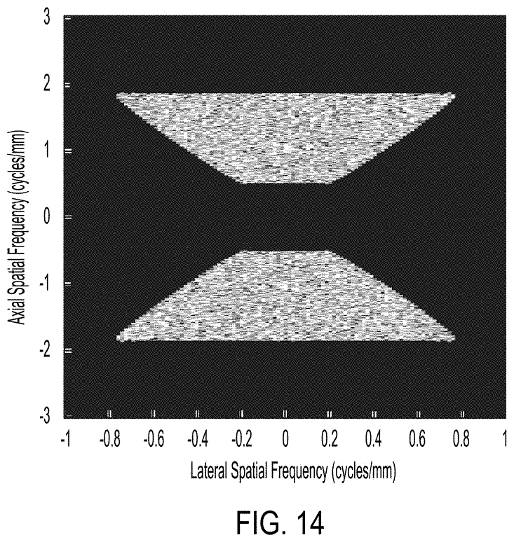

FIG. 14 shows an embodiment of a k-space representation of an estimated spatial filter for use in ultrasound imaging using coherence estimation of a beamformed signal. The horizontal axis may indicate lateral spatial frequency. The vertical axis may indicate axial spatial frequency. The axial and lateral spatial frequencies may be measured in cycles per millimeter by example.

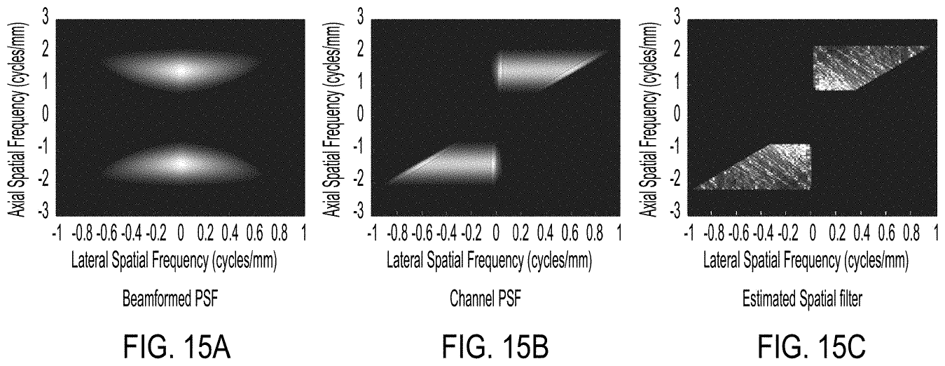

FIG. 15A shows an embodiment of a k-space representation of a beamformed channel PSF. FIG. 15B shows an embodiment of a k-space representation of a channel PSF. FIG. 15C shows an embodiment of a k-space representation of estimated spatial filters where spatial filters may be estimated from the ratio between the k-space representations of the channel PSF and the beamformed PSF, and the spatial filters may be divided into several axial segments for optimal estimation. The horizontal axes may indicate lateral spatial frequency. The vertical axes may indicate axial spatial frequency. The axial and lateral spatial frequencies may be measured in cycles per millimeter by example.

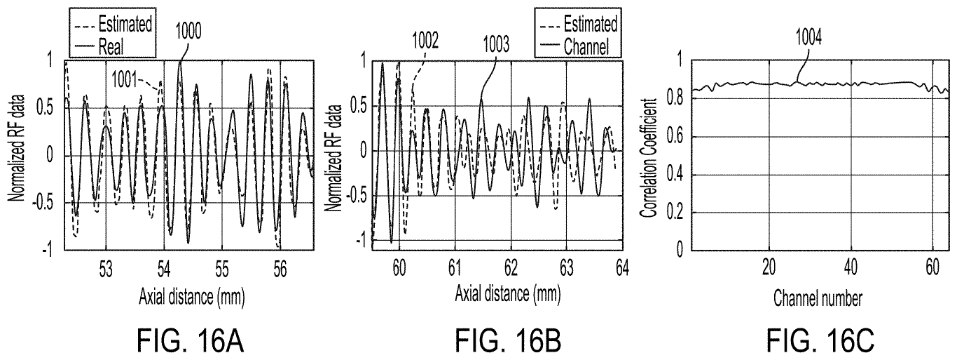

FIG. 16A is an example plot of simulated RF channel data and estimated channel data from a speckle region of an ultrasound image of anechoic cysts. The simulated RF channel data is shown by line 1000. The estimated channel data is shown by line 1001.

FIG. 16B is an example plot of simulated RF channel data and estimated channel data from a cyst region of an ultrasound image of anechoic cysts. The simulated RF channel data is shown by line 1002. The estimated channel data is shown by line 1003.

FIG. 16C is an example plot of average cross-correlation coefficients for 64 channels. The average normalized cross-correlation coefficient is 0.85 across all channels, and the cross-correlation coefficient decreases slightly for channels near the sides of the aperture as shown by line 1004 of the plot.

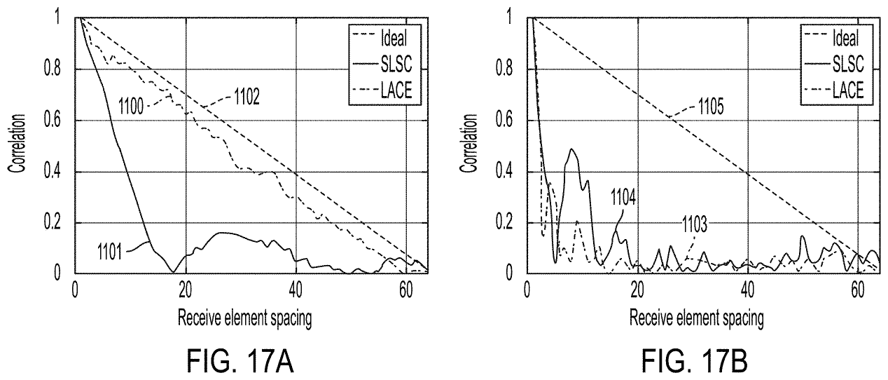

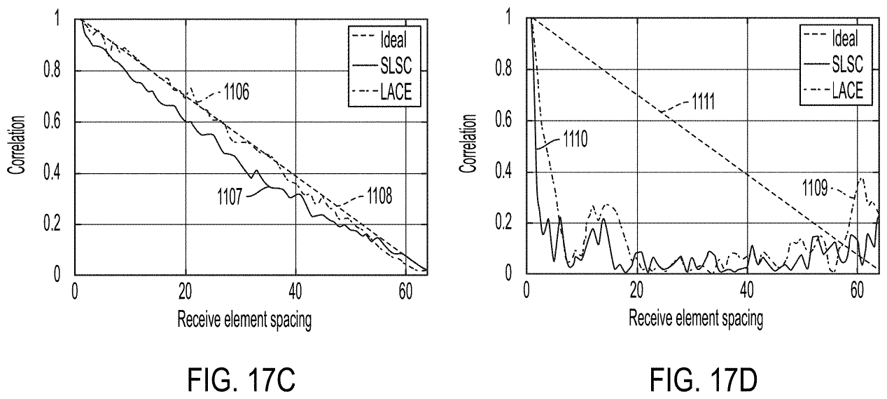

FIG. 17A is an example plot of average cross-correlation as a function of receive element spacing in a speckle region of an ultrasound image of anechoic cysts at 30 mm depth. The average cross-correlation as a function of receive element spacing is shown by line 1100 for LACE data. The equivalent cross-correlation of the SLSC data is shown by line 1101. The theoretical spatial coherence curve based on the Van Cittert Zernike theorem is shown by line 1102.

FIG. 17B is an example plot of average cross-correlation as a function of receive element spacing in a cyst region of an ultrasound image of anechoic cysts at 30 mm depth. The average cross-correlation as a function of receive element spacing is shown by line 1103 for LACE data. The equivalent cross-correlation of the SLSC data is shown by line 1104. The theoretical spatial coherence curve based on the Van Cittert Zernike theorem is shown by line 1105.

FIG. 17C is an example plot of average cross-correlation as a function of receive element spacing in a speckle region of an ultrasound image of anechoic cysts at 60 mm depth. The average cross-correlation as a function of receive element spacing is shown by line 1106 for LACE data. The equivalent cross-correlation of the SLSC data is shown by line 1107. The theoretical spatial coherence curve based on the Van Cittert Zernike theorem is shown by line 1108.

FIG. 17D is an example plot of average cross-correlation as a function of receive element spacing in a cyst region of an ultrasound image of anechoic cysts at 60 mm depth. The average cross-correlation as a function of receive element spacing is shown by line 1109 for LACE data. The equivalent cross-correlation of the SLSC data is shown by line 1110. The theoretical spatial coherence curve based on the Van Cittert Zernike theorem is shown by line 1111.

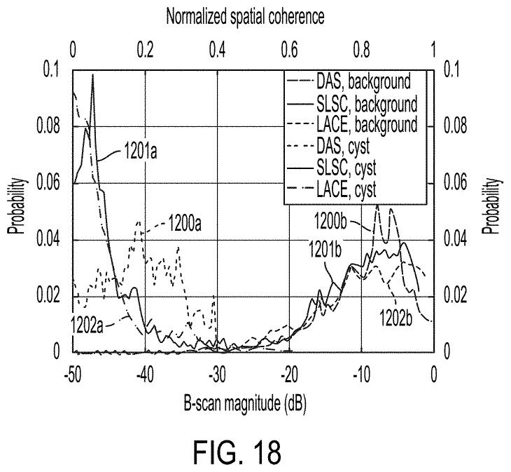

FIG. 18 is a plot of PDF of images made using DAS, SLSC, and LACE techniques to evaluate the contrast performance of the three beamforming methods. PDFs of images were plotted on the same scales used for image display. PDF of images produced using DAS is shown by lines 1200a,b. A logarithmic scale from −50 to 0 dB was used to plot lines 1200a,b. PDF of images produced using SLSC is shown by lines 1201a,b. A linear scale from 0 to 1 was used to plot lines 1201a,b. PDF of images produced using LACE is shown by lines 1202a,b. A linear scale from 0 to 1 was used to plot lines 1202a,b. Lines 1200-1202a show PDFs from a cyst region. Lines 1200-1202b show PDFs from a speckle background. As shown by the plot, the speckle region may have a higher signal amplitude than the cyst region. PDF peaks of the speckle and cyst regions being far apart may suggest a greater contrast.

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

1. A method of ultrasound imaging using coherence estimation, comprising:
receiving, by a processor, a plurality of beamformed ultrasound signals;
assembling, by the processor, the plurality of beamformed ultrasound signals into an RF signal matrix;
generating, by the processor, at least two filtered RF signal matrices using a plurality of spatial filters and the RF signal matrix;
performing, by the processor, a normalized cross-correlation on at least one pair of the filtered RF signal matrices to determine at least one cross-correlation coefficient corresponding to each pixel in an image of a target;
determining, by the processor, a coherence coefficient using the at least one cross-correlation coefficient;
constructing, by the processor, a coherence estimation image of the target using the plurality of coherence coefficients corresponding to each pixel; and
displaying, by the processor and on a display, the coherence estimation image of the target.

2. The method of claim 1, further comprising:
transmitting an ultrasound signal towards an imaging target using an array of ultrasound transducer elements;
receiving a plurality of reflected ultrasound signal using the array of ultrasound transducer elements; and
beamforming the plurality of ultrasound signals using the array of ultrasound transducer elements.

3. The method of claim 1, further comprising transforming the RF signal matrix into a k-space representation of the RF signal matrix using a frequency transform to generate the at least two RF signal matrices.

4. The method of claim 3, wherein the frequency transform is a 2D or 3D Fast Fourier Transform.

5. The method of claim 1, wherein generating the at least two filtered RF signal matrices includes: multiplying the k-space representation of the RF signal matrix by the plurality of spatial filters to generate a plurality of k-space representations of at least two filtered RF signal matrices.

6. The method of claim 5, further comprising transforming the plurality of k-space representations of the at least two filtered RF signal matrices into time domain representations of the at least two filtered RF signal matrices using an inverse frequency transform.

7. A computer readable medium storing program instructions, the program instructions comprising program instructions to configure at least one processor to:
receive a plurality of beamformed ultrasound signals and assemble the plurality of beamformed ultrasound signals into an RF signal matrix;
generate a plurality of filtered RF signal matrices by applying a plurality of spatial filters to the RF signal matrix;
perform normalized cross-correlation on a plurality of pairs of the filtered RF signal matrices to determine a plurality of cross-correlation coefficients corresponding to each pixel in the image of the target;
determine a coherence coefficient using the cross-correlation coefficients;
construct a coherence estimation image of the target using the coherence coefficients corresponding to each pixel; and
display the coherence estimation image of the target on a display.

8. The computer readable medium of claim 7, wherein the program instructions further comprise program instructions to transmit an ultrasound signal towards an imaging target using an array of ultrasound transducer elements and receive a plurality of reflected ultrasound signals using the array of ultrasound transducer elements.

9. The computer readable medium of claim 8, wherein the program instructions further comprise program instructions to beamform the plurality of ultrasound signals.

10. The computer readable medium of claim 7, wherein the program instructions further comprise program instructions to transform the RF signal matrix into a k-space representation of the RF signal matrix using a frequency transform.

11. The computer readable medium of claim 10, wherein the program instructions to generate the plurality of filtered RF signal matrices include generating a plurality of k-space representations of the plurality of filtered RF signal matrices by multiplying the k-space representation of the RF signal matrix by the plurality of spatial filters.

12. The computer readable medium of claim 11, wherein the program instructions further comprise program instructions to transform the plurality of k-space representations of the plurality of filtered RF signal matrices into time domain representations of the plurality of filtered RF signal matrices using an inverse frequency transform.

13. An ultrasound imaging system using coherence estimation, comprising:
a display screen;
an ultrasound probe having an array of transducer elements and configured to:
transmit a beamformed ultrasound signal towards an imaging target using the array of transducer elements,
receive a plurality of reflected ultrasound signals using the array of transducer elements, and
beamform the plurality of received ultrasound signals using the array of transducer elements;
a memory configured to store data; and
a processor coupled to the memory, the processor configured to:
assemble the plurality of beamformed ultrasound signals into an RF signal matrix,
generate a plurality of filtered RF signal matrices using a plurality of spatial filters and the RF signal matrix,
perform normalized cross-correlation on a plurality of pairs of the filtered RF signal matrices to determine a plurality of cross-correlation coefficients corresponding to each pixel in an image of the imaging target,
determine a coherence coefficient corresponding to each pixel in the ultrasound image of the target using the plurality of cross-correlation coefficients,
construct a coherence estimation image of the target using the coherence coefficients corresponding to each pixel, and
display the coherence estimation image on the display screen.

14. The ultrasound imaging system of claim 13, wherein the coherence estimation image has a higher contrast-to-noise ratio than an ultrasound image constructed from an amplitude of a received echo.

15. The ultrasound imaging system of claim 13, wherein the coherence estimation image has a higher signal-to-noise ratio than an ultrasound image constructed from an amplitude of a received echo.

16. The ultrasound imaging system of claim 13, wherein performance of the normalized cross-correlation is on segments of data approximately 1-4 wavelengths long in an axial direction for each filtered RF matrix in a given pair of filtered RF signals.

17. The ultrasound imaging system of claim 13, wherein forming the coherence estimation image includes using a grayscale with a coherence coefficient of 0 mapped to total black and a coherence coefficient of 1 mapped to total white.

18. The ultrasound imaging system of claim 13, wherein determining the coherence coefficient for a given pixel includes summing the cross-correlation coefficients for the given pixel.

19. The ultrasound imaging system of claim 13, wherein determining the coherence coefficient for a given pixel includes weighing and summing the cross-correlation coefficients for the given pixel.

20. The ultrasound imaging system of claim 13, wherein determining the coherence coefficient for a given pixel includes averaging the cross-correlation coefficients for the given pixel.
